(19) United States
(12) Patent Application Publication
(10) Pub. No.: US 2009/0099500 A1
(43) Pub. Date: Apr. 16, 2009

(54) MULTI-FUNCTION ELECTRODE AND USES THEREOF

(76) Inventors: Zvi Nitzan, Zofit (IL); Mordechay Moshkovich, Ra'anana (IL); Shlomo Levy, Rehovot (IL)

Correspondence Address: KENYON & KENYON LLP, 1500 K STREET N.W., SUITE 700, WASHINGTON, DC, 20005, US

(21) Appl. No.: 12/280,078

(22) PCT Filed: Feb. 28, 2007
(86) PCT No.: PCT/IL07/00265

§ 371 Date: Sep. 23, 2008

Related U.S. Application Data
(60) Provisional application No. 60/776,948, filed on Feb. 28, 2006.

(51) Int. Cl. A61N 1/30

(52) U.S. Cl. 604/20; 604/501

(57) ABSTRACT

The present invention is of an electrode comprising an electrode substrate base layer and a plurality of layers of insoluble pole electroactive substance disposed on the electrode substrate base layer, wherein each of the plurality of layers of insoluble pole electroactive substance is configured to facilitate a treatment of a condition. Further, the present invention is of use of the electrode in a currant generating treatment device.

### FIELD OF THE INVENTION

The present invention relates to a multi-layered electrode and uses thereof.

### BACKGROUND OF THE INVENTION

Examples of treatment devices include current generating treatment devices such as iontophoretic devices, which may include a main or active/donor electrode and a counter electrode. Donor electrodes as described in the background art can be in many different sizes, shapes and configurations. A typical thin and flexible electrode may include a web coated with an insoluble electroactive material composition, which comprises a layer of one insoluble electroactive material. The background art describes an electrode, which includes a layer of additional material to prevent corrosion of the donor electrode insoluble electroactive substance, such as in the case of a reactive zinc electrode.

Often when treating a condition with iontophoresis, there are a number of parameters and factors which need to be considered. These parameters may include decreasing the resistance of the body region which is to be treated in order to facilitate delivery using a minimum of current; selection of a donor electrode material according to the active drug/s to be delivered; interaction of the active drug with the electrode; reactivity of the donor electrode with other components, such as water; treatment or combination treatment with electrochemically generated ions from the electrode; competition of electrode generated ions and pretreatments of the body area. An electrode, such as those described in the background art, which includes one insoluble electroactive material may not be suitable or optimal for more than one of these parameters or conditions.

Accordingly, there is a need in the art for an electrode suitable or optimal for more than one treatment parameter or condition.

### BRIEF DESCRIPTION OF THE DRAWINGS

With reference now to the drawings in detail, it is stressed that the particulars shown, are by way of example and for the purposes of illustrative discussion of embodiments of the present invention only, and are presented for providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1a shows a schematic partial cross section of one embodiment of the electrode of the present invention;

FIG. 1b shows a schematic partial cross section of an alternative embodiment of the electrode of the present invention;

FIG. 2a shows a schematic view of one embodiment of a current generating treatment device including an electrode of the present invention;

FIG. 2b shows a schematic view of an alternative embodiment of a current generating treatment device including an electrode of the present invention;

FIG. 3 shows a flow chart of one embodiment of a method of making an electrode of the present invention;

FIG. 4 shows a flow chart of one embodiment of a method of use of an electrode according to the present invention;

FIG. 5 shows a schematic view of an electrode comprising one layer of a mixture of insoluble electroactive substances according to an embodiment of the present invention; and

FIG. 6 shows graphical representation of the discharge of a multi-layered zinc/carbon electrode compared to a one-layered carbon electrode.

### DETAILED DESCRIPTION

The present invention is of a multi-layered electrode and uses thereof. Moreover the present invention is of an electrode featuring a plurality of layers of insoluble electroactive pole materials. Furthermore, the present invention may provide a use of such an electrode in a current generating treatment device, such as an iontophoresis device.

In an embodiment, the electrode of the present invention includes a plurality of layers of insoluble electroactive pole materials. In another embodiment, the electrode of the present invention is a layered electrode, wherein each layer of insoluble electroactive material may facilitate use in the same or different treatment of a body condition. In still another embodiment, a treatment device of the present invention includes at least one of the composite electrodes of the present invention.

Embodiments of the present invention advantageously provide a multi-layered electrode, wherein each layer may provide the same or different insoluble materials and wherein each layer may have a different function or may be optimal for a different stage of treatment. The electrode may be configured as a multi-treatment electrode and eliminates the need for changing electrodes according to the stage of treatment or using a different device with a different electrode according to the stage of treatment. Moreover, the electrode may be configured as a multi-function electrode and eliminates the need for changing electrodes according to a different function according to the stage of treatment.

As used herein the term ‘treatment’ includes, but is not limited to prevention, elimination, reducing severity, alleviating direct symptoms and related symptoms and curing of a condition. The term also includes facilitating change of a condition and an improvement in appearance of a condition. The term includes a pretreatment and a post treatment, promoting delivery of a substance onto/into a body region, electrical stimulation, reducing resistance of a body region, poration of a body region, hydration and combinations thereof.

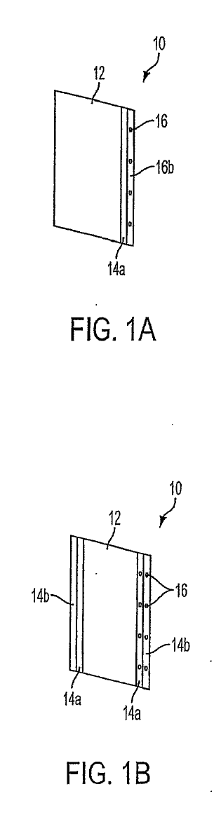

The principles and operation of an electrode and current generating treatment device according to the present invention may be better understood with reference to the figures. The figures show non-limiting embodiments of the present invention. FIG. 1a shows a partial cross section schematic view of one embodiment of the electrode of the present invention 10. As can be seen in FIG. 1a, electrode 10 features a substrate 12 and a plurality of layers of pole insoluble electro-active substance 14 (layers 14a, 14b). In some embodiments each of layers of pole insoluble electro-active substance 14 may include a binder 16 to facilitate binding of the electro-active material particles of the layers 14 together and binding the electroactive substance of the layers 14 to the electrode substrate 12. In an embodiment, wherein the layer of pole insoluble electroactive substance 14 is a foil, a conductive adhesive means may be used to bind the electroactive substance of the layer 14 to the substrate 12. In some embodiments, each of layers of pole insoluble electroactive substance may include other optional materials, such as, but not limited to, fillers, functional additives, anti-corrosive materials, anti-foaming materials, color, dye, printing stability materials and combinations thereof and any other suitable material known to one of skill in the art.

Electrode 10 may feature a layer of substrate/web 12. In some embodiments, substrate 12 is a first inner layer. The substrate base layer may be made from a material, which is sufficiently strong and stable to hold the layers of electrode 10. Optionally, the substrate can be made from at least one of or a combination of any suitable substrate material, such as, but not limited to, a conductive material, a non-conductive material, a woven material, a non-woven material, vinyl, polyester, paper, or a combination thereof. The substrate 12 may be a conductive substrate 12. In some embodiments, the conductive substrate may be electrically conductive, but is not ionically conductive.

Substrate 12 may accommodate a plurality of layers of electroactive insoluble, pole substance 14. The plurality of layers of electroactive insoluble substance 14 may be disposed on one side of the substrate as in FIG. 1a or on both sides as in FIG. 1b. Each layer of electroactive insoluble pole substance 14 can optionally be any suitable negative or positive pole electroactive insoluble substance. In an embodiment, wherein the plurality of layers of electroactive insoluble substance 14 is more than two layers, more than one layer can comprise the same electroactive insoluble substance.

As used herein the term ‘electroactive insoluble pole substance’ may include an electroactive pole substance which is sufficiently insoluble in the environment in which it is used. Examples of suitable electroactive insoluble pole substances of the layers 14 include, but are not limited to, metals and metal oxides such as, silver, silver/silver chloride, zinc, manganese dioxide, copper, magnesium, graphite, lithium, aluminum, platinum, stainless steel, tin oxide, silver oxide, gold and titanium, and/or hydrophobic polymer matrix containing a conductive filler such as metal powder/flakes, powdered graphite, carbon fibers or other known electrically conductive filler material, conductive polymer material, such as plastic and of any kind and combinations thereof.

The layer of electroactive insoluble pole substance can be applied in any suitable form including an ink, a paste, a net and a foil. In the embodiment shown in FIG. 1a, electrode 10 includes two layers of electroactive insoluble pole substance 14, a first inner layer 14a and an outer layer 14b. The invention is not limited to two layers and may include any suitable number of layers. Any suitable thickness of electroactive insoluble pole substance of the layers 14 may be used. In a non-limiting example of a thin and flexible electrode, the thickness of electroactive insoluble pole substance may be in the range of from about 1 micron to about 100 microns. The thickness of the layer of electroactive insoluble pole substance may be determined and applied according to the desired time or maximal time of treatment with that electrode substance. The time for a thin layer of insoluble pole substance 14b to be consumed will be quicker than for a thicker layer of the same substance 14b. For example in an embodiment wherein the first stage of treatment is intended as a relatively quick treatment, the thickness of the outer layer of insoluble pole substance 14b, which will be coupled to a body area, will be relatively thin.

The electroactive insoluble pole substance of each of layers 14 may be selected depending on the desired electrode properties and use of electrode 10 for each stage of treatment. In some embodiments, electroactive insoluble pole substance of each of the layers 14 may be selected such that the layers 14a and 14b do not react with each other.

The order of the layers of insoluble electroactive pole substance disposed in the electrode 10 may be chosen according to the order of electroactivity, which is based on the potentials of each electroactive pole substance. An inert electroconductive material such as graphite will not be substantially consumed. Therefore if such an inert electroconductive material is disposed as the external layer 14b, the material may not be depleted. In such an example, it is possible that with time the active material of the internal layer 14a may migrate to the graphite external layer 14b, thereby facilitating a degree of electrode function according to the identity of the internal layer 14a insoluble electroactive substance. It is also possible in such an embodiment that substantially no electrode function indicative of the internal layer 14a will be displayed.

Electroactive insoluble pole substance of the layers 14 may contain a binder material 16 or any other suitable additive. Binder material 16 may be applied with electroactive insoluble pole substance of the layers 14, in order to maintain contact between the particles and/or adhesion to substrate 12.

In some embodiments, electrode 10 may be a thin and flexible electrode. Optionally, electrodes 10 may be of any size and shape. In some embodiments, electrode 10 may be made by a printing technique. Electrodes 10 may optionally be provided as a thin sheet, or alternatively printed onto a substrate. The electrode area can be continuous, or formed as a drawing, in any shape, to provide a decorative form. In some embodiments, electrode 10 may be a perforated electrode or in a mesh form.

In some embodiments, electrode 10 has medical and cosmetic application. In some embodiments, electrode 10 may be used in a current generating treatment device, such as a dermal patch, for iontophoretic delivery of a substance or for electrical stimulation of the skin, nail, tooth, hair or any other suitable body area. In one embodiment of the present invention wherein electrode 10 is used in a dermal patch, the patch may include a power source and at least one positive electrode and at least one negative electrode, wherein at least one of the electrodes is an electrode 10 according to an embodiment of the present invention.

It is envisioned that the pole material for the outer layer, such as layer 14b, will be selected to be optimal for a first stage of treatment of a condition. For example, if the first stage is a pretreatment of a body condition, such as a wound with zinc ions, the outer layer 14b may be constructed from zinc. The thickness of the layer 14b will be predetermined in order to facilitate an adequate time for the first stage of treatment before the outer layer material is consumed exposing a second pole layer such as layer 14a. The second pole layer 14a may be selected to be optimal for a second stage of treatment. In one non-limiting example, a second stage of treatment may be delivery of an active drug with minimal delivery of competitive ions and a second pole layer 14a, suitable for such a function, may be made from graphite.

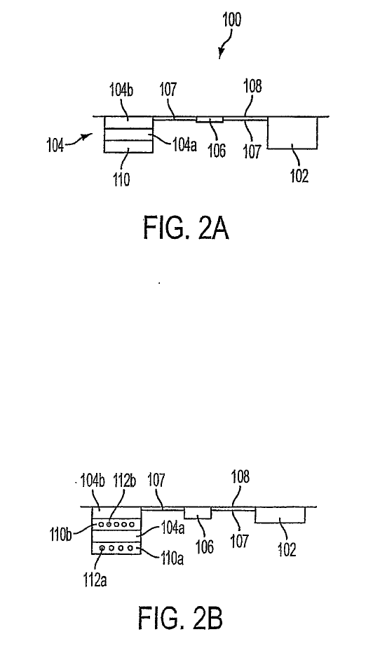

FIG. 2a shows a schematic view of a current generating treatment device including an electrode as described in FIGS. 1a or 1b according to one embodiment of the present invention. In this embodiment, device 100 may comprise at least one first electrode 102, identified as “cathode,” at least one second electrode 104, identified as “anode”, and at least one electrochemical cell 106 as the power source of device 100, wherein the cathode 102 and anode 104 are corrected to the power source 106 by suitable connection means 107 and are supported on a base layer substrate 108. Device 100 may include at least one conductive interface substance 110 for facilitating a conductive interface between the device and body area. Conductive interface substance layer 110 may include any suitable conductive composition, such as an aqueous gel, hydrogel or a conductive adhesive configured as a conductive interface between the device and body area At least one conductive interface substance may include at least one active substance, such as a drug or cosmetic substance.

In the embodiment shown in FIG. 2a, one layer of conductive interface substance 110 is disposed in contact with outer layer of pole material 104a, which is disposed on an inner layer of pole material 104b.

In an alternative embodiment shown in FIG. 2b, each insoluble electroactive layer 104a, 104b of the electrode 104 may be associated with an independent conductive interface layer 110a, 110b respectively. Each conductive interface layer 110a, 110b may include the same active drug 112a, 112b in the same or different amounts or doses. Each conductive interface layer 110a, 110b may include different drugs 112a, 112b, or one conductive interface layer may include an active drug and a second layer may not. In one non-limiting example a high dose of a drug may be disposed on the outer layer of the electrode 104a. The electrode of the present invention may be used to initially deliver a high dose of the drug. After depletion of the outer layer of the electrode 104a, the inner layer of the electrode 104b may be exposed and may be used to deliver a second drug or a lower dose of the same drug.

One of the electrodes 102 and 104 may be the main or active electrode and the other electrode the counter electrode. Defining which electrode is the main/active electrode is dependent on the charge of the ions contained in the conductive formulation 110, which is to be disposed on the main/active electrode facilitating delivery of the ions into/onto a body area. Body area may be any suitable body area, such as, but not limited to, skin, tooth, hair, mucous membrane, nail, tumor and a combination thereof. At least one of electrode 102 and 104 is a multi-layered electrode as described hereinabove for FIGS. 1a and 1b. In FIG. 2a and FIG. 2b, anode 104 is shown as the active multi-layered electrode.

In some embodiments, device 100 is a patch. Patch 100, including patch components, is thin and flexible, to suit the contour of a body area of a subject. Patch 100 can be configured to be used on any suitable area of the body, including, but not limited to, skin, mucous membrane, face, neck, arms, hands, legs, thighs, buttocks, feet, toes, fingers, nails, nail appendage, teeth, palms, soles, back, head, hair, shoulders and torso and combinations thereof.

The device 100 is configured to provide treatment to a body area. Treatment can include a plurality of treatment stages. The type of treatment may be dependent on the properties of the outer layer (exposed layer) of insoluble electroactive substance 104(a) of the active electrode. In a case wherein the insoluble electroactive substance is a reactive substance, the device 100 may facilitate electrochemical generation of electrode material ions. These ions can sometimes be advantageous and used for treatment, such as wherein the ions have therapeutic properties including antibacterial, anti-viral and anti-fungal properties, e.g. silver, copper and zinc ions. In other cases, these generated ions can compete with active formulation ions of the same charge and thereby inhibit delivery of the active compound. In one embodiment of the present invention, the combination and order of the insoluble electroactive substance layers 104(a) and 104(b) may be designed in order to control delivery of an active compound. In an example wherein the insoluble electroactive substance facilitates generation of ions, which are competition ions with the active formulation, delivery of the active formulation may be hindered. In such a way, the electrode and device can be configured to facilitate a sustained release of the active compound. In a further instance, the generated ions from the insoluble electroactive substance may be used to reduce resistance of a body area.

In an example, wherein the outer layer 104(a) is an inert material, there are minimal competitive ions with the active formulation. An inert material may be used to facilitate stability of the electrode.

The insoluble electroactive substance may have disinfectant properties and may facilitate promoting iontophoresis. An insoluble electroactive substance with high conductivity may facilitate a device delivering higher current, which may result in higher concentrations of active compound being delivered or may result in deeper penetration.

As such, the active multi-layered electrode may be designed to achieve optimal treatment according to the treatment criteria of each stage of treatment. In an example wherein a higher current is desired at the start of the treatment, a zinc outer layer 104a may be preferred. In the same treatment wherein no competitive ions are wanted, the inner layer 104b to which an active formulation is applied may be an inert material, such as graphite.

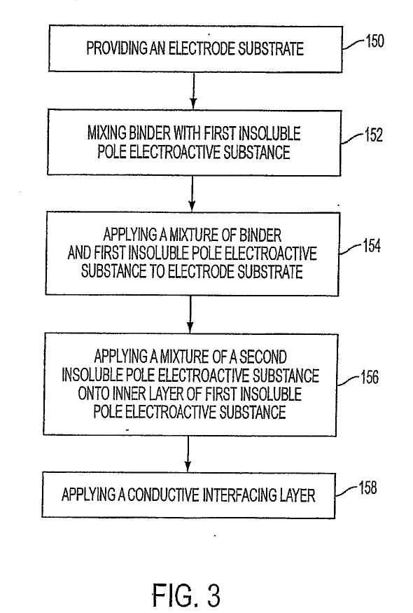

FIG. 3 shows a flow chart of a non-limiting method of making an electrode of the present invention and may include the following steps. An electrode substrate may be provided (150). Binder material may be dissolved in a suitable solvent and mixed with a first electroactive insoluble pole substance (152). The formulation of binder material and first electroactive insoluble pole substance may be applied onto the electrode substrate (154). In some embodiments, other suitable additives may be added. Any suitable method of application can be used including a printing method. The first layer of electroactive insoluble pole substance may be dried. A composition of a second electroactive pole substance can be applied onto the first layer of electroactive substance (156), which may then be dried. The second layer of electroactive substance may include a binder or any other suitable additive. Depending on how many layers of insoluble electroactive pole substance are desired, step (156) can be repeated with a different insoluble electroactive pole substance.

In some embodiments, wherein the electrode is for use in a current generating treatment device, a conductive interfacing substance, such as for example hydrogel, may then be applied onto the outer layer of electroactive insoluble pole layer (158). The conductive interfacing substance may be applied using any suitable method such as coating and printing. In some embodiments, the conductive interface substance may be applied by lamination or using a pick and place procedure. The conductive interface substance can be applied directly to electrode or can be contained in a separate compartment, which may be attached to the electrode and/or wherein the conductive interface substance is only in fluid connection on use of the electrode, such as by removing a separating means.

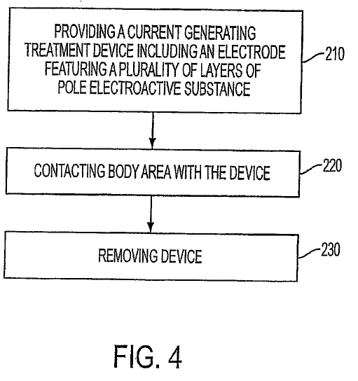

FIG. 4 is a flow chart of an exemplary method of use of an electrode according to embodiments of the present invention, wherein the electrode is included in a dermal patch. The flowchart applies to a method of use of a fully integrated patch device. A current generating treatment device, such as a device as herein described in FIG. 2a may be provided (210). In some embodiments, the device includes at least one first electrode, at least one second electrode, and at least one power source, supported on a base member in spaced relation to each other to define a gap therebetween and an integrated conductive interface substance layer, wherein the active electrode includes a plurality of layers of insoluble electroactive pole substances as described for FIGS. 1a and 1b. The patch may be configured to facilitate providing an electrical current.

The subject may contact a body area to be treated with the device (220). In some embodiments, the contact of the device with the body area facilitates current flow and promotes body area treatment of device wherein the active electrode is characterized by the exterior layer of insoluble electroactive substance.

In one non-limiting example of a device for treatment of a nail fungal infection, such as onychomycosis, the exterior insoluble electroactive substance layer may be a layer of zinc. In such an example electrochemically generated zinc ions may be delivered onto the nail to reduce the resistance of the nail as a pretreatment. The device can be configured to facilitate the pretreatment with the zinc until depletion of the zinc layer. The thickness of the layer of zinc and electrode capacity can be calculated in order to facilitate a time of pretreatment. After this predetermined time, the zinc layer may be depleted and a second inner layer of a second insoluble electroactive material is exposed and may be contacted with the body area to be treated. In one non-limiting example the second inner layer of a second insoluble electroactive substance may be an inert substance, such as graphite. An inert substance is advantageous wherein competing ions are not desired. An anti fungal active substance call be part of the second inner pole substance or alternatively can be applied to the electrode or body area after depletion of the first pole layer. The device can then be used to promote delivery of the active substance into the nail. As a result of the pretreatment with the zinc electrode, lower current and voltage may be used to promote delivery of the active substance into the nail.

The device may be removed from the body area at the end of treatment time (230).

In some embodiments, a pretreatment can be applied prior to use of the device. Non-limiting examples of pretreatments include applying a cleanser, applying a moisturizing composition, applying a formulation comprising a pharmaceutically active ingredient, applying a formulation comprising a cosmetically active ingredient, a method to enhance penetration, such as hair removal, peeling, scrubbing, electroporation, applying an agent to increase penetration, such as applying a permeation enhancer, such as urea, or a combination thereof.

In some embodiments, use of the device is a pretreatment itself, before applying a different type of treatment, such as application of a formulation.

In some embodiments, a post-treatment can be applied to the body area after application of the device. Non-limiting examples of post-treatments include applying an occlusion formulation, applying a cleanser, applying a moisturizing composition, applying a formulation comprising a pharmaceutically active ingredient, applying a formulation comprising a cosmetically active ingredient or a combination thereof.

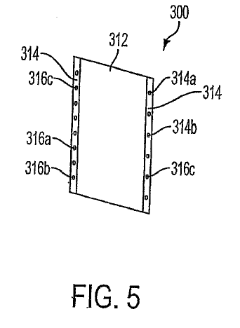

In an alternative embodiment, the electrode of the present invention may include a plurality of insoluble electroactive substances which are not disposed in layers one on top of the other, but are included in one pole layer. FIG. 5 shows a schematic representation of an electrode of the present invention according to such an embodiment. In FIG. 5, it can be seen that electrode 300 includes a layer of substrate/web 312. Substrate 312 may be a first inner layer. Disposed on substrate 312 is a layer of electroactive insoluble, pole substance 314. The layer of electroactive insoluble pole substance 314 may be disposed on both sides of the substrate or on one side of the substrate (not shown in FIG. 5). The layer of electroactive insoluble pole substance 314 may comprise a plurality of different electroactive insoluble substances, such as 314a, 314b and 314c. Non-limiting examples of suitable electroactive insoluble pole substances in the layer 314 are as listed hereinabove in the description of FIGS. 1a and 1b. The layer of electroactive insoluble pole substance can be applied in any suitable physical form.

The amount of each of the plurality of insoluble electroactive substances in the layer 314 may be determined according to the desired function of the electrode and the desired stages of treatment for which the electrode may be used. The electrode properties may be determined according to the order of consumption of each of the insoluble electroactive substances 314a, 314b and 314c, which is dependent on the electroactivity and concentration of the substances. The insoluble electroactive substance which is most active may be depleted first and the electrode 300 will exhibit properties according to this material. In such a way, an electrode comprising a layer of a mixture of zinc and carbon insoluble electroactive substances may show similar properties to a two-layered electrode of zinc and carbon.

Reference is now made to the following examples, which together with the above descriptions, illustrate the invention in a non-limiting fashion.

### EXAMPLE 1

### Preparation of a Composite Electrode Comprising Layers of Zinc and Carbon

A vinyl substrate was coated with a layer of carbon. A zinc ink was prepared and printed onto the layer of carbon using a drawdown applicator. The ink was dried at 60° C. for two hours. The electrode was then cut into an electrode of 10 mm×25 mm and hydrogel (10 mm×10 mm) was disposed on the outer zinc layer.

### EXAMPLE 2

### Testing of a Composite Electrode Comprising Layers of Zinc and Carbon

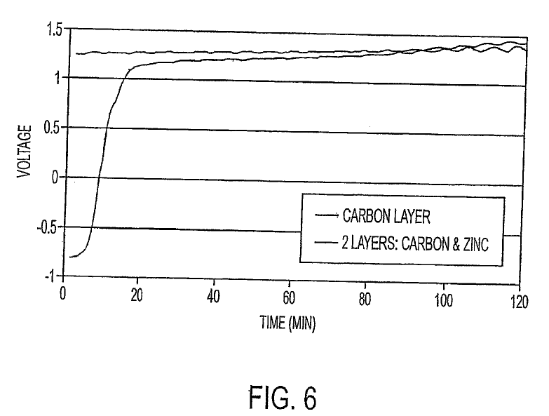

A two-layered electrode was prepared as in Example 1. The zinc/carbon electrode was discharged at 100 μA using a potentiostat/galvanostat for two hours versus a silver/silver chloride counter electrode A carbon one-layered electrode was also discharged using a potentiostat/galvanostat at 100 μA for two hours versus a silver/silver chloride counter electrode and the results compared with the two-layered electrode. FIG. 6 shows a graph of the voltage measured as a function of time of the discharge of the zinc/carbon electrode and carbon electrode. It was seen with the multi-layered zinc/carbon electrode, that from time equals zero when the zinc layer is the outer layer, the voltage is negative, indicating current generation. With increasing time, the zinc is consumed, indicated by the increase in voltage and lower current generation. At about 20 minutes, there is no current generation and the discharge values of the multi-layered electrode are the same as measured with the one-layered carbon electrode. This indicates that all the zinc of the multi-layered electrode has been depleted, leaving only the carbon layer.

The multi-layered zinc/carbon electrode displayed two different discharge conditions, each condition indicative of the outer layer insoluble electroactive material. This experiment verifies different properties of each layer of a multi-layered electrode, and the relationship with time for displaying the properties of each layer.

It is to be understood that the invention is not limited in its application to the details of construction and the arrangement of the components set forth in the description. The invention includes other embodiments and can be practiced or implemented in various ways. Also it is to be understood that the phraseology and terminology employed herein is for the purpose of description only and should not be regarded as limiting.

1. An electrode comprising:
an electrode substrate base layer; and
a plurality of layers of insoluble pole electroactive substance,
wherein the plurality of layers comprise a first layer of insoluble pole electroactive substance disposed on the substrate base layer, and at least one second layer of insoluble pole electroactive substance disposed on the first layer of insoluble pole electroactive substance, and
wherein each of the plurality of layers of insoluble pole electroactive substance is configured to facilitate a treatment of a condition.

2. The electrode of claim 1, wherein the insoluble pole electroactive substance is selected from the group consisting of metals, metal oxides, silver, silver/silver chloride, zinc, manganese dioxide, copper, magnesium, graphite, lithium, aluminum, platinum, stainless steel, tin oxide, silver oxide, gold, titanium, hydrophobic polymer matrix containing a conductive filler, conductive polymer material, plastic and combinations thereof.

3. The electrode of claim 1, wherein the plurality of layers are comprised of at least two different insoluble electroactive substances.

4. The electrode of claim 1, wherein the plurality of layers comprises at least one inner layer of insoluble pole electroactive substance and at least one external layer of insoluble pole electroactive substance and a time of a treatment is determined according to a time for depletion of an exposed external layer of insoluble electroactive substance, which is in contact with a body area.

5. The electrode of claim 1, wherein the condition is selected from at least one of a cosmetic condition and a medical condition.

6. The electrode of claim 5, wherein the medical condition is onychomycosis.

7. The electrode of claim 1, wherein the treatment is at least one of promoting delivery of a substance onto/into a body region, generating and delivering electrode pole material ions, electrical stimulation, reducing resistance of a body region, poration of a body region, hydration, pretreatment, post-treatment and a combination thereof.

8. The electrode of claim 1, further comprising at least one binder.

9. The electrode of claim 1, wherein the plurality of layers comprises a first inner layer and a second external layer and wherein the first inner layer of insoluble pole electroactive substance comprises graphite and the second external layer of insoluble pole electroactive substance comprises zinc.

10. The electrode of claim 9, wherein the zinc insoluble pole electroactive substance is configured to facilitate generation of zinc ions.

11. The electrode of claim 9, wherein the graphite insoluble pole electroactive substance is configured to facilitate delivery of an active substance.

12. The electrode of claim 4, wherein the body area is at least one of skin, tooth, hair, mucous membrane, nail, tumor and a combination thereof.

13. The electrode of claim 1 for use in a current generating treatment device.

14. (canceled)

15. The electrode of claim 1, wherein the electrode is thin and flexible.

16. A method of treating a body condition comprising: applying to a body area a current generating treatment device comprising at least one active electrode comprising an electrode substrate base layer and a plurality of layers of insoluble pole electroactive substance, wherein the plurality of layers comprise a first layer of insoluble pole electroactive substance disposed on the electrode substrate base layer, and at least one second layer of insoluble pole electroactive substance disposed on the first layer of insoluble pole electroative substance, and wherein each of the plurality of layers of insoluble pole electroactive substance is configured to facilitate a treatment of the body condition.

17. A current generating treatment device comprising at least two electrodes, wherein at least one of the at least two electrodes comprises an electrode substrate base layer and a plurality of layers of insoluble pole electroactive substance, wherein the plurality of layers comprise a first layer of insoluble pole electroactive substance disposed on the substrate base layer, and at least one second layer of insoluble pole electroactive substance disposed on the first layer of insoluble pole electroative substance and wherein each of the plurality of layers of insoluble pole electroactive substance is configured to facilitate a treatment of a condition.

18. The current generating treatment device of claim 17, further comprising a power source.

19. The current generating treatment device of claim 17, wherein a treatment is at least one of promoting delivery of a substance onto/into a body region, generating and delivering electrode pole material ions, electrical stimulation, reducing resistance of a body region, poration of a body region, hydration, pretreatment, post-treatment and a combination thereof.

20. An electrode comprising: an electrode substrate base layer; and a layer of a plurality of insoluble pole electroactive substances, wherein each insoluble pole electroactive substance is depleted according to an order of reactivity, wherein each of the plurality of insoluble pole electroactive substances is configured to facilitate a stage of treatment of a condition, and wherein the amount of each insoluble pole electroactive substance is determined according to the time of the stage of treatment.

21. The electrode of claim 20, wherein the stage of treatment is selected from the group consisting of pretreatment, post-treatment, generating and delivering electrode pole material ions, promoting delivery of a substance onto/into a body region, electrical stimulation, reducing resistance of a body region, poration of a body region, hydration and a combination thereof.
